(19) United States
(12) Patent Application Publication
(10) Pub. No.: US 2015/0011419 A1
(43) Pub. Date: Jan. 8, 2015

(54) SYSTEMS AND METHODS FOR CHARACTERIZING LUPUS ERYTHEMATOSUS

(71) Applicant: Renovar, Inc., Little Chute, WI (US)

(72) Inventors: Ganesh Vasudevan, Madison, WI (US); Huaizhong Hu, Verona, WI (US); Stuart Knechtle, Atlanta, GA (US)

(21) Appl. No.: 14/496,651

(22) Filed: Sep. 25, 2014

Related U.S. Application Data
(62) Division of application No. 12/611,711, filed on Nov. 3, 2009.
Application No. 12/611,711 is a continuation of application No. 12/409,122, filed on Mar. 23, 2009, now abandoned.
Application No. 12/409,122 is a continuation of application No. 12/188,852, filed on Aug. 8, 2008, now abandoned.
(60) Provisional application No. 60/954,656, filed on Aug. 8, 2007.

(51) Int. Cl. G01N 33/564

(52) U.S. Cl. CPC G01N 33/564; G01N 2800/104; G01N 2800/347; G01N 2333/70578
USPC 506/9; 436/501; 435/7.92

(57) ABSTRACT

The present invention provides systems and methods for characterizing biological markers in the urine of systemic lupus erythematosus (SLE) subjects. In particular, the present invention relates to the detection of cytokines and chemokines in urine of SLE subjects for determining nephritic disease states and kidney damage in SLE subjects and the efficacy of agents and interventions used to treat lupus nephritis.

### CROSS-REFERENCE TO RELATED APPLICATIONS

The present application is a divisional of U.S. patent application Ser. No. 12/611,711, filed Nov. 3, 2009, which is a continuation of abandoned U.S. patent application Ser. No. 12/409,122, filed Mar. 23, 2009, which is a continuation of abandoned U.S. patent application Ser. No. 12/188,852, filed Aug. 8, 2008, which claims priority to expired U.S. Provisional Patent Application No. 60/954,656, filed Aug. 8, 2007, each of which are herein incorporated by reference in its entirety.

### FIELD OF THE INVENTION

The present invention provides systems and methods for characterizing biological markers in the urine of systemic lupus erythematosus (SLE) subjects. In particular, the present invention relates to the detection of cytokines and chemokines in urine of SLE subjects for determining nephritic disease states and kidney damage in SLE subjects and the efficacy of agents and interventions used to treat lupus nephritis.

### BACKGROUND OF THE INVENTION

Systemic lupus erythematosus (SLE or lupus) is a chronic autoimmune disease that is potentially debilitating and sometimes fatal as the immune system attacks the body's cells and tissue, resulting in inflammation and tissue damage. SLE can affect any part of the body, but most often harms the heart, joints, skin, lungs, blood vessels, liver, kidneys and nervous system. The course of the disease is unpredictable, with periods of illness (called flares) alternating with remission. Lupus can occur at any age, but is most common in women, and is treatable symptomatically, mainly with corticosteroids and immunosuppressants, though there is currently no cure. The prevalence in the United States had been estimated as approximately 500,000 but a recent survey commissioned by the Lupus Foundation of America suggested a prevalence of as many as 2,000,000. For example, a recent study identified a prevalence of 500 per 100,000 (1:200) in women residing in the area surrounding Birmingham, Ala. The prognosis for patients with SLE has greatly improved over the last few decades with at least 80-90% of all patients surviving ten years.

Studies have focused on the cytokine profile in peripheral mononuclear blood cells of patients with SLE. However, since organ involvement in SLE is highly variable, the study of peripheral blood cells is not representative of the local immunopathogenesis at specific sites. In the case of lupus nephritis, renal biopsies are often required in the management of SLE. There is some controversy as to the best timing since many rheumatologists recommend empirical therapy of initial episodes of nephritis with corticosteriods. For refractory, recently relapsed, or frequently relapsed renal disease a biopsy is useful, especially to identify candidates for cytotoxic therapy. However, a non-invasive method for determining lupus nephritis flares and kidney damage is preferential to an invasive biopsy procedure.

As such, what are needed are methods for performing non-invasive diagnostic procedures on SLE patients determining the presence or absence of nephritis and the presence or absence of associated kidney damage in these patients. Such methods would provide a more rapid, cost effective, and less traumatic alternative to both clinician and patient with regards to tracking and determining lupus nephritis and kidney damage from the disease.

### SUMMARY OF THE INVENTION

The present invention provides systems and methods for characterizing biological markers in the urine of systemic lupus erythematosus (SLE) subjects. In particular, the present invention relates to the detection of cytokines and chemokines in urine of SLE subjects for determining nephritic disease states and kidney damage in SLE subjects and the efficacy of agents and interventions used to treat lupus nephritis.

Certain illustrative embodiments of the invention are described below. The present invention is not limited to these embodiments.

Accordingly, in some embodiments, the present invention provides systems (e.g., kits, reaction mixtures, reagents, etc.) and methods for providing biomarkers and/or combinations of biomarkers for use in determining the presence or absence of increased lupus nephritis activity (e.g., a flare) and for determining the presence or absence of kidney damage is such patients, comprising: providing a urine sample from a subject, wherein the subject is suspected of having a lupus flare or lupus associated kidney damage, providing reagents for detection and quantification of one or more compounds, including but not limited to, adiponectin, monocyte chemotactic protein-1 (MCP-1), osteoprotegerin, urokinase-type plasminogen activator receptor (uPAR), and insulin-like growth factor binding protein-2 (IGFBP-2) and detecting the presence of the compounds in the urine sample using the reagents. In some embodiments, the detecting the presence of the compound in the urine sample comprises detecting the amount of the compound in the urine sample. In other embodiments, the method further comprises providing a sample additive composition comprising a high concentration salt buffer, wherein the salt buffer, when mixed with an equal volume of urine and reagents for detection and quantification, provides a total concentration (e.g., salt from the buffer in addition to salt found in the urine) of salt of 200-600 mM in the mixture.

In some embodiments, the present invention provides methods of detecting a disorder of the kidney associated with lupus, comprising providing a urine sample from a subject, wherein the subject is suspected of having lupus nephritis, providing reagents for quantification of one or more compounds from the list comprising MCP-1, IGFBP-2, osteoprotegerin, and uPAR, and quantifying the amount of the compounds in the urine sample using the reagents. In some embodiments the amount of compounds quantified in the urine sample is at least 50 pg/ml, at least 100 pg/ml, or at least 200 pg/ml, indicating a disorder of the kidney associated with lupus. In some embodiments, the subject is determined to be at risk for a disorder of the kidney associated with lupus, based on the amount of said compounds detected in said urine. In some embodiments, the subject is determined to suffer from a disorder of the kidney associated with lupus based on the amount of said compounds detected in said urine. In some embodiments, the amount of one or more compounds in said urine sample is quantified to be at a threshold over background, indicating a disorder of the kidney associated with lupus (e.g. 2-fold over background, 5-fold over background, 10-fold over background, 20-fold over background, or 50-fold over background).

In some embodiments, the compound is not fragmented (e.g. a full length compound). In other embodiments the compound is a fragment. In further embodiments, the reagents comprise reagents for performing an immunoassay. In preferred embodiments the immunoassay comprises an ELISA, radioimmunoassay, automated immunoassay, cytometric bead assay, and/or immunoprecipitation assay. In some embodiments, the reagents comprise reagents for performing a fluorescently activated cell-sorting assay. In some embodiments, the method further comprises the step of determining a treatment course of action based on the presence or absence of a lupus flare or lupus associated kidney damage. In some embodiments, the method further comprises the step of determining a treatment course of action based on detecting a disorder of the kidney associated with lupus.

The present invention further provides a method of diagnosing a lupus flare and/or lupus associated kidney damage, comprising providing a urine sample from a subject, providing reagents for detection and quantification of one or more compounds including, but not limited to, adiponectin, IGFBP-2, MCP-1, osteoprotegerin, and uPAR, detecting the presence of the one or more compounds from the list, and diagnosing a lupus flare and/or kidney damage in the subject based on the results of the detecting. In other embodiments, the detecting the presence of the compound in the urine sample comprises detecting the amount of the compound in the urine sample. In some embodiments, the method further comprises the step of determining a treatment course of action based on the diagnosis of a lupus flare and/or lupus associated kidney disease.

In further embodiments, the present invention provides a kit, comprising reagents and/or other components (e.g., buffers, instructions, solid surfaces, containers, software, etc.) sufficient for, necessary for, or useful for detecting one or more compounds including, but not limited to, adiponectin, IGFBP-2, MCP-1, osteoprotegerin, and uPAR, uPAR, instructions for using said reagents for detecting the presence of one or more of said compounds, and instructions for using said detecting the amount of said one or more compounds in said urine sample for detecting a disorder of the kidney associated with lupus. In some embodiments, the kit further comprises a sample additive composition comprising a high concentration salt buffer, wherein said salt buffer, when mixed with an equal volume of urine and reagents for detection and quantification, provides a concentration of salt of 200-600 mM in the mixture. In some embodiments, the instructions comprise instructions required by the United States Food and Drug Administration for use in in vitro diagnostic products.

In some embodiments, the treatment course of action comprises the administration of therapeutic agents. In some embodiments, the treatment course of action comprises a surgical procedure. In additional embodiments the surgical procedure comprises renal transplantation. In further embodiments the treatment course of action comprises dialysis. In some embodiments the dialysis is hemodialysis. In other embodiments the dialysis is peritoneal dialysis. In other embodiments, the treatment course of action comprises continued monitoring.

The present invention additionally provides a method of determining a treatment course of action, comprising providing a urine sample from a subject, wherein the subject is suspected of having a lupus flare and/or kidney damage, and detecting the presence of or amount of a cytokine and/or chemokine in the urine sample using the reagents; and determining a treatment course of action based on the detecting. In some embodiments, the treatment course of action comprises continued monitoring. The present invention is not limited to the detection of a particular cytokine and/or chemokine. Any suitable cytokine and/or chemokine is contemplated including, but not limited to, adiponectin, IGFBP-2, MCP-1, osteoprotegerin, and uPAR. In some embodiments, the cytokine and/or chemokine is a full-length cytokine and/or chemokine. In other embodiments, the cytokine and/or chemokine is a fragment of a full-length cytokine and/or chemokine. The present invention is not limited to a particular assay. In some embodiments, the reagents comprise reagents for performing an immunoassay. For example, any suitable immunoassay is contemplated including, but not limited to, ELISA, radio-immunoassay, automated immunoassay, cytometric bead assay, and immunoprecipitation assay. In some embodiments, the ELISA is a quantitative ELISA assay. In other embodiments the assay is a LUMINEX bead assay. In further embodiments, the assay is a protein microarray.

The present invention also provides a method of screening compounds, comprising providing a sample from a subject, wherein the subject is suspected of having a lupus flare and/or lupus associated kidney damage; an assay with reagents for detection and quantification of a cytokine and/or chemokine; and one or more test compounds; and administering the test compound to the subject; and detecting the amount of cytokine and/or chemokine in the sample using the reagents. The present invention is not limited to a particular sample type. Any bodily fluid including, but not limited to, blood, urine, serum, and lymph may be utilized. In some preferred embodiments, the sample is a urine sample. In some embodiments, the test compound is a drug. In some embodiments, the method further comprises the step of determining the efficacy of the drug based on the detecting. The present invention is not limited to the detection of a particular cytokine and/or chemokine. Any suitable cytokine and/or chemokine is contemplated including, but not limited to, adiponectin, IGFBP-2, MCP-1, osteoprotegerin, and uPAR.

### DESCRIPTION OF THE FIGURES

FIG. 1 shows fold over background of selected urine chemokines and cytokines of a subject experiencing a lupus nephritic flare compared to a SLE subject that is not experiencing a flare.

### DEFINITIONS

To facilitate an understanding of the present invention, a number of terms and phrases are defined below:

As used herein, the term “fluorescently activated cell sorting assay” (FACS) refers to any assay suitable for use in cell sorting techniques (e.g., flow cytometry) that employs detection of fluorescent signals.

As used herein, the terms “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

As used herein, “cytokine” refers to any of a class of immunoregulatory substances (for example, lymphokines) that are secreted by cells of the immune system. As used herein, “cytokine-related compound” refers to any of a class of substances that are functionally linked to one or more cytokines, for example, adhesion molecules, selectins, integrins, chemokines, and chemokine receptors. In some embodiments as used herein, “cytokines” includes, but is not limited to osteoprotegerin (OPG).

As used herein, “chemokines” are cytokines characterized, for example, by their ability to induce directed migration of leukocytes, leukocyte activation and effector function (e.g., chemotactic cytokines). As used herein, “chemokines” can be divided into, for example, four branches (C, CC, CXC, and CX3C) based upon the position of the first two cysteine residues in a four-cysteine motif in their primary amino acid sequence. As used herein, chemokines are also classified by their binding characteristics as ligands (L), for example CL, CCL, CXCL and CX3CL. As used herein, “chemokines” are further characterized based on whether they are inflammatory or homeostatic.

As used herein, “diagnosing a lupus flare” and “diagnosing lupus related kidney damage” refers to, for example, the detection, identification, monitoring, and screening of lupus nephritis. In some embodiments the diagnosis uses only the assays of the present invention. In other embodiments, assays of the present invention are used for diagnosis of a lupus flare and/or lupus related kidney damage in combination with other indices of kidney function including, for example, patient signs and symptoms, tests of general kidney function, for example, serum creatinine and blood urea nitrogen (BUN), or urinalysis, or tests of specific disorders of the kidney, for example, kidney biopsy, urine RNA levels, urine DNA levels, and other urinary markers. In some embodiments, assays of the present invention are performed in a health care facility laboratory. In other embodiments, assays of the present invention are performed in a reference clinical laboratory. In further embodiments, assays of the present invention are performed at the patient's residence by the patient, a caregiver, or health care provider. In some embodiments of the present invention, diagnosing a lupus flare and/or kidney damage is based on detecting at least one compound from the list comprising adiponectin, IGFBP-2, MCP-1, osteoprotegerin, and uPAR.

As used herein, “detecting the presence” and “detecting the amount” of said compounds refer to a qualitative or quantitative measure of the compound in the urine of a subject.

As used herein, the term “determining a treatment course of action” as in “determining a treatment course of action based on said diagnosis of a lupus flare and/or lupus related kidney damage” refers to the choice of treatment administered to a patient. For example, if a patient is found to be at increased risk of a developing a flare or kidney damage, therapy may be started, increased, or changed from one treatment type (e.g., pharmaceutical agent, surgery) to another. In some embodiments, the treatment course of action is “continued monitoring” in which treatment is maintained but the levels of cytokines, cytokine-related compounds and chemokines measured in the patients urine is monitored regularly (e.g., using the diagnostic methods of the present invention). In other embodiments, the “treatment course of action” as used herein, comprises use of the results of the cytokine, cytokine-related compound and chemokine assays of the present invention as indicators of the need for additional tests, for example, an imaging scan, biopsy, etc.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence (that is, the “full size” sequence) minus one amino acid.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the terms “protein microarray” and “protein chip” refer to protein-detecting molecules immobilized at high density on a substrate, and probed for various biochemical activities. (See, for example: Zhu H and Snyder M, “Protein chip technology”, Current Opinion in Chemical Biology 7: 55-63, 2003; Cutler P, “Protein arrays: The current state of the art”, Proteomics 3; 3-18, 2003; and MacBeath G, “Protein microarrays and proteomics”, Nature Genetics Supplement 32: 526-532, 2002, each of which is incorporated herein by reference in its entirety).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (for example, a lupus flare or lupus related kidney damage). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include urine and blood products, such as plasma, serum and the like. Such examples are not however to be construed as limiting the sample types applicable to the present invention.

### DETAILED DESCRIPTION OF THE INVENTION

In some embodiments, the present invention provides non-invasive methods of correlating the presence of certain cytokines, cytokine-related compounds and/or chemokines in urine (or other body fluids) with SLE flares and lupus related kidney damage. The methods are a significant improvement over invasive biopsy in terms of decreased cost and physical trauma to a patient. The methods of the present invention provide the further advantage of facilitating home testing by patients.

### I. Detection of Cytokines, Cytokine-Related Compounds and Chemokines in Urine

In some embodiments, the present invention provides methods of predicting, determining, and diagnosing a lupus flare by detecting cytokines, cytokine-related compounds and chemokines in urine. The present invention is not limited to a particular detection assay. The description below provides non-limiting examples of suitable cytokines, cytokine-related compounds and chemokines and detection methods. The present invention further provides kits for use in detecting cytokines, cytokine-related compounds and chemokines in urine.

### A. Urinary Cytokines, Cytokine-Related Compounds and Chemokines

The present invention provides methods of detecting cytokines, cytokine-related compounds and chemokines in urine. The urinary cytokines, cytokine-related compounds and chemokines of the present invention are correlated with the presence or absence of a lupus flare and/or lupus related kidney damage. In some embodiments, the presence of the peptides or an increased amount of the peptides is indicative of flares or damage. In other embodiments, increased urinary cytokines, cytokine-related compounds and chemokines are correlated with increased risk of experiencing a lupus flare or developing kidney damage. In some embodiments, the amount of urinary cytokine, cytokine-related compound and chemokine is quantitated. In some embodiments, a quantitative level of urinary cytokine, cytokine-related compound and chemokine is determined that is indicative of an increased risk of experiencing a lupus flare or developing kidney damage. In other embodiments, the level of cytokine, cytokine-related compound and chemokine is correlated with a functioning level of a drug (e.g., the correct amount of a functional drug).

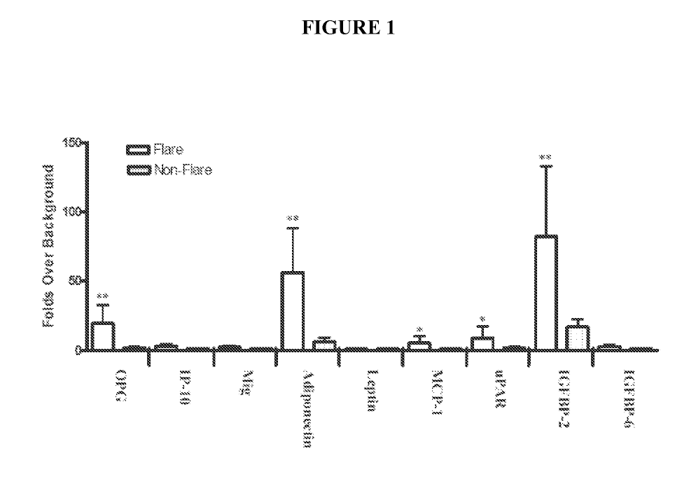

In some embodiments, the cytokines, cytokine-related compounds and chemokines are one or more of adiponectin, IGFBP-2, MCP-1, osteoprotegerin (OPG), and uPAR (FIG. 1).

In some embodiments, two or more (e.g., 3 or more, 4 or more, etc.) cytokines, cytokine-related compounds and chemokines are detected to provide a risk assessment. The presence of each marker may provide a more definitive answer than the analysis of any single marker alone.

In some embodiments, certain threshold levels of a particular marker are detected. If the threshold level is reached, risk of experiencing a lupus flare, disorder of the kidney associated with lupus, and/or kidney damage is observed. In some embodiments, the concentration threshold level of a particular marker is ≧50 pg/ml, ≧100 pg/ml, or ≧200 pg/ml. In some embodiments, the FOZ (fold over zero), or fold over background threshold level of a particular marker is ≧2, ≧3, ≧5, ≧10, ≧20, or ≧50.

### B. Detection Methods

The present invention provides methods for detecting the presence of cytokines, cytokine-related compounds and chemokines in a urine sample. In some embodiments, a full-size cytokine, cytokine-related compound or chemokine polypeptide is detected. In other embodiments, a fragment or a portion of a cytokine, cytokine-related compound or chemokine polypeptide is detected. In preferred embodiments, the present invention additionally provides methods of quantifying the amount of a cytokine, cytokine-related compound and chemokine in urine. The present invention is not limited to a particular detection assay. In some embodiments detection is, for example, fluorescent detection, spectrometric detection, chemiluminescent detection, matrix assisted laser desorption-time-of flight (MALDI-TOF) detection, high pressure liquid chromatographic detection, charge detection, mass detection, radio frequency detection, and light diffraction detection. Exemplary detection assays are described herein.

In some embodiments, cytokines, cytokine-related compounds and chemokines are detected by binding of a capture molecule specific for the protein (for example, an aptamer, or an antibody in an immunoassay). The present invention is not limited to a particular capture molecule or antibody. Any capture molecule or antibody (e.g., monoclonal or polyclonal) that detects cytokines, cytokine-related compounds and chemokines may be utilized. Exemplary methods for the generation of antibodies are described below.

Antibody binding is detected by techniques known in the art. For example, in some embodiments, antibody binding is detected using a suitable technique, including but not limited to, radio-immunoassay, ELISA (enzyme-linked immunosorbant assay), “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay.

In some preferred embodiments, a quantitative ELISA assay is utilized (See e.g., U.S. Pat. Nos. 5,958,715, and 5,484,707, each of which is herein incorporated by reference). In some preferred embodiments, the quantitative ELISA is a competitive ELISA. In a competitive ELISA, the wells of a microtiter plate are first coated with a fusion protein comprising all or a fragment of the cytokine, cytokine-related compound or chemokine. The sample to be tested is added to the plate along with an antibody that is specific for the cytokine, cytokine-related compound or chemokine. The cytokine, cytokine-related compound or chemokine in the urine sample competes for binding to the antibody with the immobilized peptide. The plate is washed and the antibody bound to the immobilized cytokine, cytokine-related compound or chemokine polypeptide is then detected using any suitable method (e.g., a secondary antibody comprising a label or a group reactive with an enzymatic detection system). The amount of signal is inversely proportional to the amount of cytokine, cytokine-related compound or chemokine polypeptide present in the urine sample (e.g., a high signal is indicative of low amounts of cytokine, cytokine-related compound or chemokine polypeptide being present in the urine).

In some embodiments, an automated detection assay is utilized. Methods for the automation of immunoassays include, but are not limited to, those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a diagnosis and/or prognosis based on the level of cytokine, cytokine-related compound or chemokine polypeptide in the urine is utilized. In other embodiments, the immunoassay described in U.S. Pat. Nos. 5,789,261, 5,599,677 and 5,672,480, each of which is herein incorporated by reference, is utilized.

In still other embodiments, a protein microarray or protein chip array assay is utilized for detection (See e.g., U.S. Pat. No. 6,197,599, herein incorporated by reference). In such an assay, proteins (e.g., antibodies specific for a cytokine, cytokine-related compound or chemokine polypeptide) are immobilized on a solid support such as a chip. A urine sample suspected of containing the cytokine, cytokine-related compound or chemokine polypeptide is passed over the solid support. Bound cytokine, cytokine-related compound or chemokine polypeptides are then detected using any suitable method. In some embodiments, detection is via surface plasmon resonance (SPR) (See e.g., WO 90/05305, herein incorporated by reference). In SPR, a beam of light from a laser source is directed through a prism onto a biosensor consisting of a transparent substrate, usually glass, which has one external surface covered with a thin film of a noble metal, which in turn is covered with an organic film that interacts strongly with an analyte, such as a biological, biochemical or chemical substance. The organic film contains antibodies (e.g., specific for a cytokine, cytokine-related compound or chemokine polypeptide of the present invention), which can bind with an analyte (e.g., chemokine) in a sample to cause an increased thickness, which shifts the SPR angle. By either monitoring the position of the SPR angle, or the reflectivity at a fixed angle near the SPR angle, the presence or absence of an analyte in the sample can be detected.

In other embodiments, The PROTEINCHIP (Ciphergen Biosystems, Fremont, Calif.) is utilized for detection. The PROTEINCHIP system uses SELDI (Surface-Enhanced Laser Desorption/Ionization) technology to perform the separation, detection and analysis of proteins at the femptomole level directly from biological samples (See e.g., U.S. Pat. No. 6,294,790 and U.S. Patent Application US20010014461A1, each of which is herein incorporated by reference. In the PROTEINCHIP technology, proteins of interest (e.g., cytokine, cytokine-related compound or chemokine polypeptides) are captured on the PROTEINCHIP Array (e.g., via a bound antibody) directly from the original source material. The chip is washed to remove undesired materials and bound proteins are detected using SELDI.

In some embodiments, a cytometric bead array assay is used (Quantum Plex kit, Bangs Laboratories; Cytometric Bead Array kit, BD Biosciences). These systems allow for multiple analyte detection with small volume samples. In other embodiments, a LUMINEX bead assay is used.

The present invention is not limited to the detection of cytokines, cytokine-related compounds and chemokines in urine. Any bodily fluid that contains elevated levels of cytokine, cytokine-related compound and chemokine correlated with a kidney disorder may be utilized, including, but not limited to, blood, serum, lymph, and saliva.

In some particularly preferred embodiments, a combination of several cytokines, cytokine-related compounds or chemokines are detected simultaneously in urine samples. In some embodiments, the present invention provides a fluorescently activated cell sorting (FACS) method for the simultaneous detection of multiple cytokines, cytokine-related compounds or chemokines. In some embodiments, the method uses fluorescence dye labeled beads that can detect multiple (e.g., at least 3) cytokines, cytokine-related compounds or chemokines in one assay. As the chemokine concentration increases, the mean fluorescence intensity for each group of beads increases. This correlation between the chemokine concentration and the mean fluorescence establishes the basis for this FACS quantitative method. Results demonstrate a quantitative assay for the simultaneous detection of multiple cytokines, cytokine-related compounds and chemokines.

The present invention is further not limited to the direct detection of cytokine, cytokine-related compound and chemokine polypeptides. The present invention contemplates the detection of correlated polypeptides or compounds (e.g., cytokine, cytokine-related compound and chemokine DNA, mRNA, metabolites, etc.). In still further embodiments, the present invention provides methods of detecting the interaction of cytokines, cytokine-related compounds and chemokines with cytokine, cytokine-related compound and chemokine receptors).

### C. Kits

In some embodiments, the present invention provides kits for the detection of cytokines, cytokine-related compounds and chemokines. In some embodiments, the kits contain antibodies specific for cytokines, cytokine-related compounds and chemokines in addition to detection reagents, buffers or devices. In preferred embodiments, the kits contain all of the components necessary or sufficient to perform a detection assay, including all controls, directions for performing assays, and any necessary hardware or software for analysis and presentation of results.

In some embodiments, the kits contain an assay in a test strip format. In such embodiments, the detection reagent (e.g., antibody), as well as any control or secondary antibodies, are affixed to a solid support. In some embodiments, the solid support is a test strip suitable for dipping into a solution of urine (See e.g., U.S. Pat. Nos. 6,352,862, 6,319,676, 6,277,650, 6,258,548, and 6,248,596, each of which is herein incorporated by reference).

In some embodiments, the kits are marketed as in vitro diagnostics. The marketing of such kits in the United States requires approval by the Food and Drug Administration (FDA). The FDA classifies in vitro diagnostic kits as medical devices. The 510(k) regulations specify categories for which information should be included.

### II. Patient Care

The present invention further provides methods of providing test kits to patients in a variety of settings. The test kits of the present invention are suitable for use in both clinical and home testing settings. In preferred embodiments, test kits are approved for sale as in vitro diagnostics as described above.

### A. Home Testing

In some embodiments, the present invention provides kits for home testing. In preferred embodiments, the kits are approved as in vitro diagnostics for home use under guidelines as described above. Patients may use home test kits to monitor for a lupus flare, the progression of an ongoing flare, or the risk of developing kidney damage. In some embodiments, test kits for home use are qualitative rather than quantitative. For example, in some embodiments, the test registers a positive result if urine levels of cytokines, cytokine-related compounds and chemokines are above a pre-determined level (e.g., a threshold level such as a fold increase over background) or increase over time. In other embodiments, the tests are quantitative (e.g., utilizing the quantitative methods described above).

For example, in some embodiments, patients with lupus monitor urine levels of cytokines, cytokine-related compounds and chemokines for an impending lupus flare, or an ongoing flare, or potential kidney damage from an ongoing or cumulative flares. In preferred embodiments, patients conduct serial monitoring (e.g., from once a day to once a month or every several months) to screen for early signs of a flare or kidney damage. In preferred embodiments, patients whose urine levels of cytokines, cytokine-related compounds and chemokines are above a pre-determined level (or register a positive result in a quantitative assay) are instructed to seek medical advice.

In other embodiments, the test kits are utilized by patients, caregivers or health care providers at the patient's residence to monitor the effectiveness of a drug. For example, in some embodiments, a patient who is taking one or more drugs following a diagnosis of SLE monitors levels of cytokines, cytokine-related compounds and chemokines on a regular basis (e.g., from once a day to once a month or every several months). If a patient's levels of cytokines, cytokine-related compounds or chemokines are above a pre-determined level (or registers a positive result in a quantitative assay), it may be indicative of a lupus flare or kidney damage caused by lack of an effective level of a drug. Such patients are advised to schedule a follow up with a caregiver (e.g., to adjust the medication levels, or switch to a different drug).

### B. Clinical-Based Testing

In other embodiments, testing is performed in a clinical (e.g., hospital or clinic) setting. In such embodiments, testing is generally ordered and interpreted by a physician or other clinician. In some embodiments, testing is carried out by a lab technician (e.g., in an in-house or external clinical lab). In preferred embodiments, clinical testing utilizes a quantitative assay for detection of cytokines, cytokine-related compounds and chemokines. In some embodiments, testing is utilized to determine the likelihood of kidney damage in a patient with SLE. In other embodiments, testing is utilized to monitor organ function in a subject who has recovered from a lupus flare and/or kidney damage. In still further embodiments, testing is utilized to monitor the effectiveness of a medication. In some embodiments, the urinary cytokine, cytokine-related compound or chemokine test is used to complement a biopsy and/or serum creatinine (Cr), and to monitor response to therapy. In a preferred embodiment, the urinary cytokine, cytokine-related compound and chemokine test is used as a reference parameter in lieu of a biopsy.

The urinary cytokine, cytokine-related compound and chemokine test of the present invention is simple to conduct and rapid, making it suitable for clinical use. In some embodiments, testing is utilized as a follow up to home testing by a patient (e.g., when cytokines, cytokine-related compound and chemokines levels are elevated or the patient has other clinical signs or symptoms of a lupus flare or kidney damage). Based on the result of the clinical testing, the appropriate intervention is taken (e.g., including, but not limited to, an increase or decrease in levels of drug therapy, initiation of drug therapy, change in drug therapy, termination of therapy, surgery, further testing, or continued monitoring).

### C. Home Collection/Clinic Testing

In still further embodiments, testing is provided by a clinical lab. For example, in some embodiments, the patient collects a urine specimen and transports the specimen to a clinical lab (e.g., by mail or in person). The clinical lab then reports the result to the patient. In other embodiments, the patient provides a sample at a clinical lab, the sample is analyzed, and the results are returned to the patient. The patient then decides, based on the level of cytokines, cytokine-related compounds and chemokines in the urine (or the presence or absence of a positive result in a qualitative assay) whether or not to contact a physician for follow up care. In other embodiments, testing is provided by a clinical lab that reports the results to the patient's physician. The patient's physician then reports the results to the patient, either maintaining existing treatment, augmenting treatment, and/or changing treatment altogether.

### III. Antibodies

The present invention provides isolated antibodies. In preferred embodiments, the present invention provides monoclonal antibodies that specifically bind to an isolated polypeptide comprised of at least five amino acid residues of a cytokine, cytokine-related compound or chemokine. These antibodies find use in the diagnostic methods described herein. In other embodiments, commercially available antibodies are utilized (e.g., available from any suitable source including, but not limited to, R & D System, Minneapolis, Minn.).

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a cytokine or chemokine) For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared, and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide-activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cytokine, cytokine-related compound or chemokine polypeptide (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### IV. Drug Screening

In some embodiments, the present invention provides drug-screening assays (e.g., to screen for drugs effective in treating lupus flares and/or lupus related kidney damage). The screening methods of the present invention utilize the detection of cytokines, cytokine-related compounds and chemokines. For example, in some embodiments, the present invention provides methods of screening for compounds that alter (e.g., increase or decrease) the expression of cytokines, cytokine-related compounds and chemokines. In some embodiments, the levels of cytokines, cytokine-related compounds and chemokines are detected (e.g., using a method described herein) in a subject that has undergone administration of a candidate compound. The increased levels of cytokines, cytokine-related compounds and chemokines are indicative of a candidate compound that is not preventing a lupus flare. Conversely, preferred candidate compounds are those that normalize cytokine, cytokine-related compound and chemokine levels.

In some embodiments, drug screening assays are performed in animals. Any suitable animal may be used including, but not limited to, baboons, rhesus or other monkeys, mice, or rats. Animal models of SLE or SLE related kidney damage are generated (e.g., by the administration of compounds that trigger renal failure), and the effects of candidate drugs on the animals are measured. In preferred embodiments, kidney disorders in the animals are measured by detecting levels of cytokines, cytokine-related compounds and chemokines in the urine of the animals. The level of cytokines, cytokine-related compounds and chemokines may be detected using any suitable method, including, but not limited to, those disclosed herein.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of detecting and treating a disorder of the kidney associated with systemic lupus erythematosus, comprising:
a) obtaining a urine sample from a subject, having systemic lupus erythematosus;
b) having said urine sample tested to quantify the amount of osteoprotegerin in said urine sample; and
c) initiating or increasing therapy for said disorder of the kidney associated with systemic lupus erythematosus if said amount of osteoprotegerin is above a threshold level.

2. The method of claim 1, wherein said threshold level of osteoprotegerin is at least 50 pg/ml.

3. The method of claim 1, wherein said threshold level of osteoprotegerin is at least 100 pg/ml, indicating a disorder of the kidney associated with lupus.

4. The method of claim 1, wherein said threshold level of osteoprotegerin is at least 200 pg/ml.

5. The method of claim 1, wherein said threshold level of osteoprotegerin is at least 2-fold over background.

6. The method of claim 1, wherein said threshold level of osteoprotegerin is at least 5-fold over background.

7. The method of claim 1, wherein said threshold level of osteoprotegerin is at least 10-fold over background.

8. The method of claim 1, wherein said threshold level of osteoprotegerin is at least 20-fold over background.

9. The method of claim 1, wherein said threshold level of osteoprotegerin at least 50-fold over background.

10. The method of claim 1, wherein said osteoprotegerin is a full length osteoprotegerin.

11. The method of claim 1, wherein said osteoprotegerin is a fragment of osteoprotegerin.

12. The method of claim 1, wherein said urine sample is tested by performing an immunoassay.

13. The method of claim 12, wherein said immunoassay is selected from the group consisting of an ELISA, radio-immunoassay, automated immunoassay, cytometric bead assay, and immunoprecipitation assay.

14. The method of claim 1, wherein said urine sample is tested by performing a fluorescently activated cell sorting assay.

15. The method of claim 1, further comprising the step of determining a treatment course of action based on said detecting a disorder of the kidney associated with lupus.

16. The method of claim 1 further comprising: having said urine sample tested to quantify MCP-1, IGFBP-2, and/or uPAR.
